(19) United States
(12) Patent Application Publication
(10) Pub. No.: US 2016/0000849 A1
(43) Pub. Date: Jan. 7, 2016

(54) COMPOSITION FOR REMEDYING OR TREATING RHEUMATOID ARTHRITIS AND OSTEOARTHRITIS

(71) Applicant: HANPOONG PHARM & FOODS CO., LTD., jeonju-si Jeonllabuk-do (KR)

(72) Inventors: Hyoung-kwon CHO, Jeonju-si (KR); Ki-young JUN, Jeonju-si (KR); Jun-kyoung LEE, Imsil-gun (KR); Chang-hyun JEON, Wanju-gun (KR); Dae-sung KIM, Iksan-si (KR); Erisa BYUN, Jeonju-si (KR); Jin-ju YU, Wanju-gun (KR); Young-bae KWON, Jeonju-si (KR); Jung-kee KWON, Damyang-gun (KR); Dae-ki KIM, Iksan-si (KR); Se-gun KIM, Donghae-si (KR); Hyeon-ju JEONG, Iksan-si (KR); Eun-joo LEE, Daejeon (KR)

(21) Appl. No.: 14/770,168

(22) PCT Filed: Dec. 13, 2013
(86) PCT No.: PCT/KR2013/011597

§ 371 Date: Aug. 25, 2015

(30) Foreign Application Priority Data
Mar. 29, 2013 (KR) 10-2013-0034040

(51) Int. Cl. A61K 36/732; A61K 36/714; A61K 36/185

(52) U.S. Cl. CPC A61K 36/732; A61K 36/185; A61K 36/714

(57) ABSTRACT

The present invention relates to the composition according to the present invention, which has not only the effect of suppressing inflammation and pain, which are the common symptoms of acute and chronic arthritis, but also the effect of alleviating movement disorders, which are the symptoms of chronic arthritis.

### TECHNICAL FIELD

The present invention relates to a composition for alleviating or treating rheumatoid arthritis and osteoarthritis.

In particular, the present invention relates to a composition for alleviating or treating rheumatoid arthritis and osteoarthritis, which has not only the effect of suppressing inflammation and pain, which are the common symptoms of acute and chronic arthritis, but also the effect of alleviating movement disorders, which are the symptoms of chronic arthritis.

Furthermore, the present invention relates to a composition for alleviating or treating rheumatoid arthritis and osteoarthritis, which has the above effects and is also not cytotoxic to cartilage tissue and synovial tissue, thereby enabling the joint structure to be protected and maintained.

Moreover, the present invention relates to a composition for alleviating or treating rheumatoid arthritis and osteoarthritis, which is based on the results of tests conducted using an acute arthritis animal model, a chronic rheumatoid arthritis animal model, and a chronic osteoarthritis animal model.

### BACKGROUND ART

Arthritis is a disease that causes inflammation and pain, and can be chiefly divided into osteoarthritis (degenerative arthritis) and rheumatoid arthritis.

Osteoarthritis, also called degenerative arthritis, occurs mainly in the elderly without a specific organic cause, and will cause movement disorders, such as gait disturbances, due to the deformation of the joint structure when it progresses to a chronic state.

Osteoarthritis causes damage to the bond and ligament of the joint mainly due to progressive damage to joint cartilage or degenerative changes in joint cartilage, thereby causing inflammation and pain.

The mechanism of osteoarthritis is as follows. As osteoarthritis progresses, the production of inflammatory cytokines such as TNF-α, IL-1 and the like increases, the secretion of matrix metalloproteinases (MMPs), such as collagenase, stromelysin and the like, increases, thereby causing the breakdown of joint cartilage. MMPs induce IL-1, TNF-α and the like, which affect tissues, such as muscle, tendon, ligament and the like, resulting in severe pain.

In this case, MMPs (matrix metalloproteinases) are major factors that are involved in cartilage damage, and can be divided into collagenases, gelatinases, stromelysins, and membrane-type MMPs and several other groups of MMPs.

Rheumatoid arthritis is an inflammatory disease characterized by polyarthritis, and autoimmunity is known as the major cause of rheumatoid arthritis.

The symptoms of rheumatoid arthritis are as follows. As inflammation occurs in synovial membrane tissue, macrophages, dendritic cells, T lymphocytes, B lymphocytes and the like migrate to synovial tissue, and as a result, the amount of joint fluid increases so that the joint swells to cause pain.

When the hyperplasia of inflammatory synovial tissue occurs while such inflammation continues, the bone and cartilage are destroyed, and as a result, the joint structure is deformed and movement disorders occur.

According to the results of studies, it is known that inflammatory cytokines in rheumatoid arthritis patients produce collagenase and neutral protease in synovial fibroblasts, and the produced enzymes degrade collagen and proteoglycan to destroy joint cartilage.

In summary, when arthritis is developed, joint inflammation, fever and edema resulting from inflammation, and joint pain occur, and cartilage and bone tissues are destroyed while the symptoms of arthritis progress, resulting in the deformation of the joint tissue and in movement disorders.

Therefore, there is an urgent need for a drug that can suppress inflammation and pain, which are the common symptoms of acute and chronic arthritis, and can also alleviate or treat movement disorders that are chronic symptoms. In addition, there is an urgent need for a drug that has the above-described effects and that is also not cytotoxic to cartilage tissue and synovial tissue, thereby allowing the joint tissue to be protected and maintained.

Hereinafter, natural materials used in the present invention will be described in brief.

Kalopanacis Cortex is the bark of Kalopanax pictus Nakai, which is a deciduous tree belonging to the family Araliaceae, or the bark of Kalopanax pictus Nakai var. chinensis Nakai, Kalopanax pictus Nakai var. magnificus Nakai and Kalopanax pictus Nakai var. maximowiczii Nakai, which belong to the same family.

It is known that Kalopanacis Cortex has a tannin content of 13-30%, and contains glucose, kalotoxin, kalosaponin, liriodendrin, hederagenin, arabinose, benzoic acid, amino acid, d-mannitol and polyacetylene, and the main components thereof are saponin, phenolic glycosides, etc.

Kalopanacis cortex is widely used for neuralgia, joint pain, lumbago, syphilis, diarrhea, toothache, diabetes, tonic activity and the like in folk remedies.

Chaenomelis Fructus is the ripe fruit of Chaenomeles sinensis Koehne or Chaenmeles speciosa Nakai, which is a deciduous shrub belonging to the family Rosaceae.

Chaenomelis Fructus is known to contain saponin, malic acid, tartaric acid, citric acid, vitamin C, flavonoids, tannin, etc.

In Chinese medicine, Chaenomelis Fructus is recognized as a drug that harmonizes the spleen and stomach and removes dampness. Accordingly, it is used as an effective drug for acute gastrointestinal diseases, beriberi, muscle pain, arthritis and neuralgia. Furthermore, Chaenomelis Fructus is known to have antitussive and expectorant activities and to be effective against pneumonia, bronchitis and the like.

Raphani Semen is the seed of annual or biennial Raphanus sativus L. belonging to the family Cruciferae or other plants belonging to the same family.

Raphani Semen contains fatty oils and essential oils. The essential oils contain methylthiole and the like, and the fatty oils contain large amounts of erucic acid, linoleic acid, and an ester of glycerinsinapic acid.

Raphani Semen has the effects of allowing “qi” to flow and relieving indigestion, and thus is used mainly for abdominal dropsy, belching, gastric hyperacidity, diarrhea, etc. It is known to be effective in treating a loss of appetite, loosening old phlegm and easing chronic coughs.

Atractylodis Rhizoma is the rhizome of Atractylodes lancea DC. or A. chinensis Koidzumi, which is a perennial plant belonging to the family Compositae.

Atractylodis Rhizoma contains 5-9% essential oil as a medically effective ingredient, and the main components of the essential oil include atractylol, hinesol, β-eudesmol, and the like. It is slightly bitter and slightly hot in taste and warming in nature.

Atractylodis Rhizoma has a remarkable effect of strengthening the stomach and exhibits an excellent effect on the treatment of digestive disorders caused by the excessive accumulation of dampness in the stomach and intestines, as well as on the treatment of gastrointestinal functional disorders. In addition, it contains a large amount of vitamin A, and thus is used for nyctalopia.

Aconiti Lateralis Preparata Radix is the processed daughter root of Aconitum carmichaeli Debx. that is a perennial plant belonging to the family Ranunculaceae.

As ingredients of Aconiti Lateralis Preparata Radix, alkaloids, including hypaconitine, aconitine, mesacontine, talatisamine and the like, are known. In addition, it is known that aconitine, mesaconitine, hypaconitine and jesaconitine, which are diterpene-based alkaloids, have strong toxicity.

Aconiti Lateralis Preparata Radix enhances the yang-qi balance of the kidneys and has the effect of strengthening the heart. It is used for yang-qi weakness caused by various chronic diseases, general paralysis, paralysis of extremities, cold, chronic ulcers, intestinal convulsions, diarrhea, etc. It was reported to have pharmacological effects, including myocardial contraction, blood pressure increase, anti-inflammation, pain alleviation, anti-coldness, immune enhancement, pituitary and adrenal cortex stimulation, blood pressure lowering, etc.

The applicant has developed a composition for alleviating or treating rheumatoid arthritis and osteoarthritis, which is prepared using Kalopanacis Cortex, Chaenomelis Fructus, Raphani Semen, Atractylodis Rhizoma, and Aconiti Lateralis Preparata Radix as described above. Prior arts related to these materials are described in brief below.

First, Korean Patent Application Publication No. 10-2011-0016825 discloses a composition for preventing or treating arthritis, which contains a mixed ingredient of Schizandra chinensis, Scutellaria root and Kalopanacis Cortex as an active ingredient. Furthermore, Korean Patent Application Publication No. 10-2003-0091405 discloses a composition for preventing or treating arthritis, which contains an ethyl acetate soluble extract of Kalopanacis Cortex as an active ingredient.

Furthermore, Korean Patent Application Publication No. 10-2011-0038631 discloses a composition for cartilage regeneration, pain suppression and edema suppression, which contains, as an active ingredient, extracts of Chaenomelis Fructus, Achyranthis radix, Acanthopanax senticosus, Cinnamomum cassia bark, Gentianae macrophyllae radix, Clematis chinensis root, Angelica root, Cnidium officinale root, Gastrodia elata, Carthamus tinctorius, Dipsasi radix and Saposhnikovia divaricata root.

Moreover, Korean Patent Application Publication No. 10-2009-0074475 discloses a method of preparing an herbal medicinal drug for treatment of joints using 12-13 natural materials, including Atractylodis Rhizoma.

In summary, although Kalopanacis Cortex, Chaenomelis Fructus and Atractylodis Rhizoma are described as materials for the prevention or treatment of arthritis, they are used chiefly by mixing them with other materials and extracting the mixture. For Raphani Semen and Aconiti Lateralis Preparata Radix that are used in the present invention, a prior art related to arthritis has not been found.

### DISCLOSURE

### Technical Problem

The present invention is intended to provide a composition for alleviating or treating rheumatoid arthritis and osteoarthritis, which has not only the effect of suppressing inflammation and pain, which are the common symptoms of acute and chronic arthritis, but also the effect of alleviating movement disorders, which are the symptoms of chronic arthritis.

The present invention is also intended to provide a composition for alleviating or treating rheumatoid arthritis and osteoarthritis, which has the above effects and is also not cytotoxic to cartilage tissue and synovial tissue, thereby enabling the joint structure to be protected and maintained.

The present invention is also intended to provide a composition for alleviating or treating rheumatoid arthritis and osteoarthritis, which is based on the results of tests conducted using an acute arthritis animal model, a chronic rheumatoid arthritis animal model and a chronic osteoarthritis animal model.

### Technical Solution

In order to accomplish the above objects, the present invention provides a composition for alleviating or treating rheumatoid arthritis and osteoarthritis, which contains, as an active ingredient, an extract of 350-450 parts by weight of Kalopanacis Cortex, 350-450 parts by weight of Chaenomelis Fructus, 50-150 parts by weight of purified Aconiti Lateralis Preparata Radix, 150-250 parts by weight of Raphani Semen, and 50-150 parts by weight of Atractylodis Rhizoma.

In the present invention, the extract may be: 1) an extract obtained by mixing a first extract, prepared by extracting 350-450 parts by weight of Kalopanacis Cortex, 350-450 parts by weight of Chaenomelis Fructus and 50-150 parts by weight of purified Aconiti Lateralis Preparata Radix, with a second extract prepared by extracting 150-250 parts by weight of Raphani Semen and 50-150 parts by weight of Atractylodis Rhizoma; or 2) a mixed extract of 350-450 parts by weight of Kalopanacis Cortex, 350-450 parts by weight of Chaenomelis Fructus, 50-150 parts by weight of purified Aconiti Lateralis Preparata Radix, 150-250 parts by weight of Raphani Semen and 50-150 parts by weight of Atractylodis Rhizoma.

In the present invention, the composition is characterized in that it is not cytotoxic to chondrocytes and synovial cells, and has anti-inflammatory and pain-relieving effects on arthritis patients and the effect of alleviating movement disorders in chronic arthritis patients.

In the present invention, an extraction solvent for the first extract may be 40-60 wt % alcohol, and an extraction solvent for the second extract may be water.

In the present invention, the number of times each of the first extract, the second extract and the mixed extract is extracted may be two or more.

The present invention may also provide a dry extract formulation for alleviating or treating rheumatoid arthritis and osteoarthritis, which is prepared by concentrating and drying the above-described extract.

The present invention may also provide a pharmaceutical formulation for alleviating or treating rheumatoid arthritis and osteoarthritis, which is prepared by adding a pharmaceutically acceptable additive to the above-described extract.

### Advantageous Effects

The composition according to the present invention has the effect of alleviating or treating rheumatoid arthritis and osteoarthritis.

In particular, the composition according to the present invention has remarkable effects based on the results of tests conducted using an acute arthritis animal model, a chronic rheumatoid arthritis animal model and a chronic osteoarthritis animal model.

The composition according to the present invention has not only the effect of suppressing inflammation and pain, which are the common symptoms of acute and chronic arthritis, but also the effect of alleviating movement disorders, which are the symptoms of chronic arthritis.

The composition according to the present invention has the effect of being not cytotoxic to cartilage tissue and synovial tissue, thereby enabling the joint structure to be protected and maintained.

### DESCRIPTION OF DRAWINGS

FIG. 1 shows the results of measuring cytotoxicity in a group treated with glucosamine-HCl, a group treated with glucosamine-sulfate and a group treated with celecoxib;

FIG. 2 shows the results of measuring cytotoxicity in a group treated with HPL-1;

FIG. 3 shows cytotoxicity in synovial sw982 cells incubated with HPL-1 for 24 hours;

FIG. 4 shows cytotoxicity in synovial sw982 cells incubated with HPL-1 for 48 hours;

FIG. 5 shows the results of measuring cytotoxicity in a chloroform fraction and ethyl acetate fraction of HPL-1;

FIG. 6 is a graph showing the anti-inflammatory effect of HPL-1 upon induction of acute arthritis;

FIG. 7 is a graph showing the pain-relieving effect of HPL-1 in a paw withdrawal latency test for evaluating mechanical allodynia upon induction of acute arthritis;

FIG. 8 is a graph showing the pain-relieving effect of HPL-1 in a paw withdrawal latency test for evaluating mechanical hyperalgesia upon induction of acute arthritis;

FIG. 9 is a graph showing the pain-relieving effect of HPL-1 in a paw withdrawal latency test for evaluating thermal hyperalgesia upon induction of acute arthritis;

FIG. 10 is a graph showing the anti-inflammatory effect of HPL-1 upon induction of chronic rheumatoid arthritis;

FIG. 11 is a graph showing the pain-relieving effect of HPL-1 in a paw withdrawal latency test for evaluating mechanical allodynia upon induction of chronic rheumatoid arthritis;

FIG. 12 is a graph showing the pain-relieving effect of HPL-1 in a paw withdrawal latency test for evaluating mechanical hyperalgesia upon induction of chronic rheumatoid arthritis;

FIG. 13 is a graph showing the pain-relieving effect of HPL-1 in a paw withdrawal latency test for evaluating thermal hyperalgesia upon induction of chronic rheumatoid arthritis;

FIG. 14 is a graph showing the effect of alleviating gait disturbances upon induction of chronic osteoarthritis; and

FIG. 15 is a graph showing the effect of alleviating gait disturbances upon induction of chronic osteoarthritis.

### BEST MODE

The terms and words used in the present specification and the claims should not be interpreted as being limited to typical meanings or dictionary definitions, but should be interpreted as having meanings and concepts consistent with the technical scope of the present invention, based on the principle that an inventor can appropriately define the concept of the terms to describe his or her invention in the best way.

Accordingly, the embodiments and examples described in the specification and the particulars illustrated in the drawings are merely the most preferable exemplary embodiments of the present invention, but do not represent all the technical spirit of the present invention. Therefore, it should be understood that various equivalents and modifications that can replace the above embodiments, examples and particulars may be present at the time at which the present application is filed.

### Embodiment 1: Composition for Alleviating or Treating Rheumatoid Arthritis and Osteoarthritis

In an embodiment of the present invention, a method for preparing a composition for alleviating or treating rheumatoid arthritis and osteoarthritis is as follows.

1) 350-450 parts by weight of Kalopanacis Cortex, 350-450 parts by weight of Chaenomelis Fructus, 50-150 parts by weight of purified Aconiti Lateralis Preparata Radix, 150-250 parts by weight of Raphani Semen, and 50-150 parts by weight of Atractylodis Rhizoma are prepared.

Kalopanacis Cortex, Chaenomelis Fructus and purified Aconiti Lateralis Preparata Radix are mixed with one another, and then extracted once with 40-60 wt % alcohol, thereby preparing a first extract. In this case, the extraction is performed for 2-4 hours.

Raphani Semen and Atractylodis Rhizoma are mixed with each other, and then extracted once with water, thereby preparing a second extract. In this case, the extraction is performed for 2-4 hours.

The first and second extracts are filtered through a 25 μm filter and then mixed with each other to prepare a mixture.

A composition for alleviating or treating rheumatoid arthritis and osteoarthritis is prepared using the mixture as an active ingredient. The mixture may be concentrated under reduced pressure at 60° C. or lower and dried to provide a dry extract formulation.

It will be apparent that design conditions may be changed so that the first extract or the second extract are prepared by performing extraction twice or more and the mixture is treated by processes different from the reduced-pressure concentration and drying processes to provide various formulations. In addition, in the process of preparing the first extract, extraction with other kinds of organic solvent fractions may further be performed after extraction with 40-60 wt % alcohol.

2) Based on 350-450 parts by weight of Kalopanacis Cortex, 350-450 parts by weight of Chaenomelis Fructus, 50-150 parts by weight of purified Aconiti Lateralis Preparata Radix, 150-250 parts by weight of Raphani Semen and 50-150 parts by weight of Atractylodis Rhizoma are prepared.

Kalopanacis Cortex, Chaenomelis Fructus, purified Aconiti Lateralis Preparata Radix, Raphani Semen and Atractylodis Rhizoma are mixed with one another, and then extracted once with 40-60 wt % alcohol or water, thereby preparing a mixed extract. In this case, the extraction is performed for 2-4 hours.

A composition for alleviating or treating rheumatoid arthritis and osteoarthritis is prepared using the mixed extract as an active ingredient. The mixed extract may be filtered through a 25 μm filter and then concentrated under reduced pressure at 60° C. or lower and dried to provide a dry extract formulation.

It will be apparent that design conditions may be changed so that the mixed extract is prepared by performing extraction twice or more and the mixed extract is treated by processes different from the reduced-pressure concentration and drying processes to provide various formulations. In addition, in the process of preparing the mixed extract, extraction with other kinds of organic solvent fractions may further be performed after extraction with 40-60 wt % alcohol.

3) A pharmaceutically acceptable carrier, excipient or diluent is added to the composition for alleviating or treating rheumatoid arthritis and osteoarthritis, prepared in the above method 1) or 2), thereby providing the composition in a pharmaceutical unit dosage form.

In this case, examples of the carrier, excipient or diluent include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxylbenzoate, talc, magnesium stearate, and mineral oil.

Furthermore, the pharmaceutical dosage form may be used in the form of a pharmaceutically acceptable salt, and may also be used alone or in combination with other pharmaceutically active compounds.

Furthermore, a pharmaceutical composition including the composition for alleviating or treating rheumatoid arthritis and osteoarthritis may be formulated with commonly used diluents or excipients, such as fillers, extenders, binders, wetting agents, disintegrants, surfactants, etc.

Moreover, the pharmaceutical dosage form may be prepared in oral dosage forms, including powders, granules, tablets, capsules, suspensions, emulsions, syrup and aerosol, as well as suppositories and sterile injectable solutions.

Solid formulations for oral administration may be prepared by adding at least one excipient, for example, starch, calcium carbonate, sucrose, lactose, gelatin or the like, to the extract. In addition to simple excipients, lubricants such as magnesium stearate or talc may also be used.

Formulations for parenteral administration include sterilized aqueous solutions, non-aqueous solutions, suspensions, emulsions, freeze-dried preparations, and suppositories.

As non-aqueous solvents or suspending agents, propylene glycol, polyethylene glycol, plant oils such as olive oil, injectable esters such as ethyl oleate, and the like can be used.

As the base of the suppositories, witepsol, Macrogol, Tween 61, cacao butter, laurin fat, glycerogelatin and the like can be used.

The preferred dose of the composition for alleviating or treating rheumatoid arthritis and osteoarthritis according to the present embodiment varies depending on the patient's condition and bodyweight, the severity of the disease, the patient's age and gender, the form of drug, and the route and period of administration, but can be appropriately selected by those skilled in the art.

The composition for alleviating or treating rheumatoid arthritis and osteoarthritis according to the present embodiment may be administered to mammals, including rats, mice, livestock and humans, by various routes. All routes of administration can be contemplated and include. For example, the composition for alleviating or treating rheumatoid arthritis and osteoarthritis according to the present embodiment may be administered via oral, tissue, rectal, intravenous, intramuscular or subcutaneous routes.

### PREPARATION EXAMPLE 1

### Composition for Alleviating or Treating Rheumatoid Arthritis and Osteoarthritis

In a preparation example of the present invention, a composition for alleviating or treating rheumatoid arthritis and osteoarthritis was prepared as follows.

Kalopanacis Cortex, Chaenomelis Fructus and purified Aconiti Lateralis Preparata Radix were mixed with one another at a weight ratio of 4:4:1 and then extracted twice with 50% alcohol to prepare a first extract. In this case, the extraction time was 3 hours or more for each extraction.

Raphani Semen and Atractylodis Rhizoma were mixed with each other at a weight ratio of 2:1, and then extracted twice with water to prepare a second extract. In this case, the extraction time was 3 hours or more for each extraction.

The first and second extracts were filtered through a 25 μm filter and then mixed with each other to prepare a mixture.

The mixture was concentrated under reduced pressure at 60° C. or lower and dried to prepare a dry extract.

### TEST EXAMPLE 1

### Cytotoxicity in Chondrocytes

It is to be noted that Test Examples 1 to 7 were performed using the mixture prepared in Preparation Example 1 above as a sample.

In addition, it is to be noted that ‘HPL-1’ or ‘HPL-01,’ described in Test Examples 1 to 7 and shown in the figures, is the name temporarily designated by the applicant during the tests, and refers to the mixture prepared in Preparation Example 1.

1-1: Test Process

The stabilities of glucosamine-HCl, glucosamine-sulfate, celecoxib and HPL-1 were analyzed by an MTT assay using normal 293T cells, rat chondrocytes and human chondrocytes (HTB-94).

1-2: Test Results

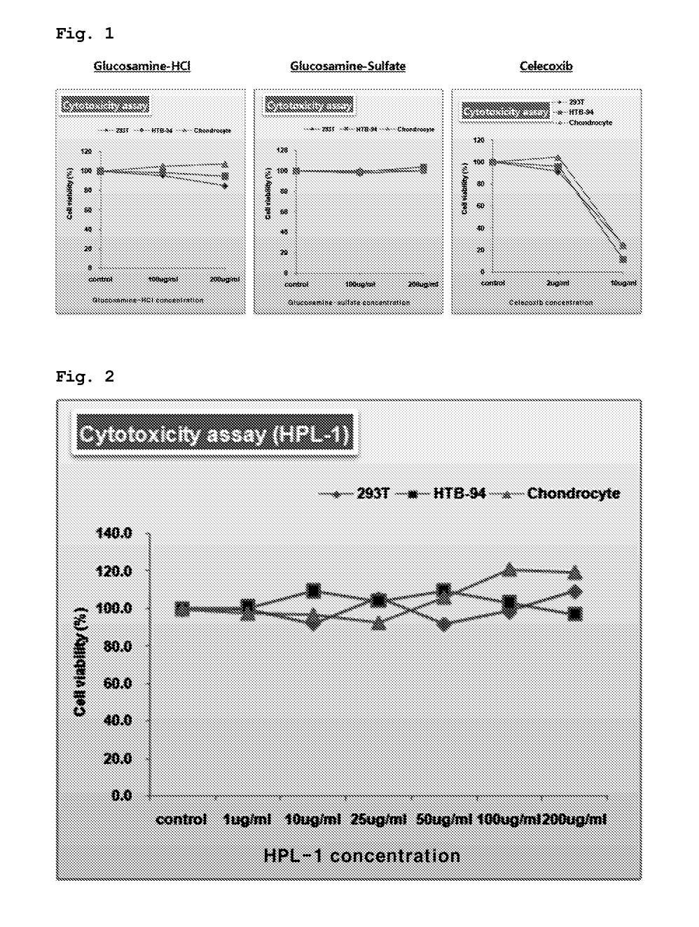

FIG. 1 shows the results of measuring cytotoxicity in a group treated with glucosamine-HCl, a group treated with glucosamine-sulfate and a group treated with celecoxib.

FIG. 2 shows the results of measuring cytotoxicity in a group treated with HPL-1.

As can be seen in FIGS. 1 and 2, no cytotoxicity was observed in all the group treated with glucosamine-HCl, the group treated with glucosamine-sulfate and the group treated with HPL-1, and cytotoxicity was observed in the celecoxib-treated group at a concentration of 10 μg/mL or more.

In particular, in the group treated with HPL-1 prepared in Preparation Example 1 of the present invention, cytotoxicity did not appear even at a concentration of up to 200 μg/ml.

### TEST EXAMPLE 2

### Protective Effect on Synovial Cells

2-1: Test Process

(1) In order to examine the effect of HPL-1 on the proliferation of human synovial sw982 cells, a CCK-8 assay was performed. The CCK-8 assay was performed according to a conventional method.

Synovial cells treated with at various concentrations (1, 10, 50, 100, 200 and 500 μg/ml) of HPL-1 were incubated for 24 hours and 48 hours, and the results of the incubation were compared with those of a control.

(2) In order to evaluate the accurate ability of HPL-1 to inhibit the activity of synovial cells, a chloroform (CHCl₃) fraction and ethyl acetate (EtOAc) fraction of HPL-1 were taken and incubated at varying concentrations (10, 100, 200 and 400 μg/ml) for 24 hours and 48 hours because disturbing factors can be removed by a combined material. The results of the incubation were compared.

2-2: Test Results

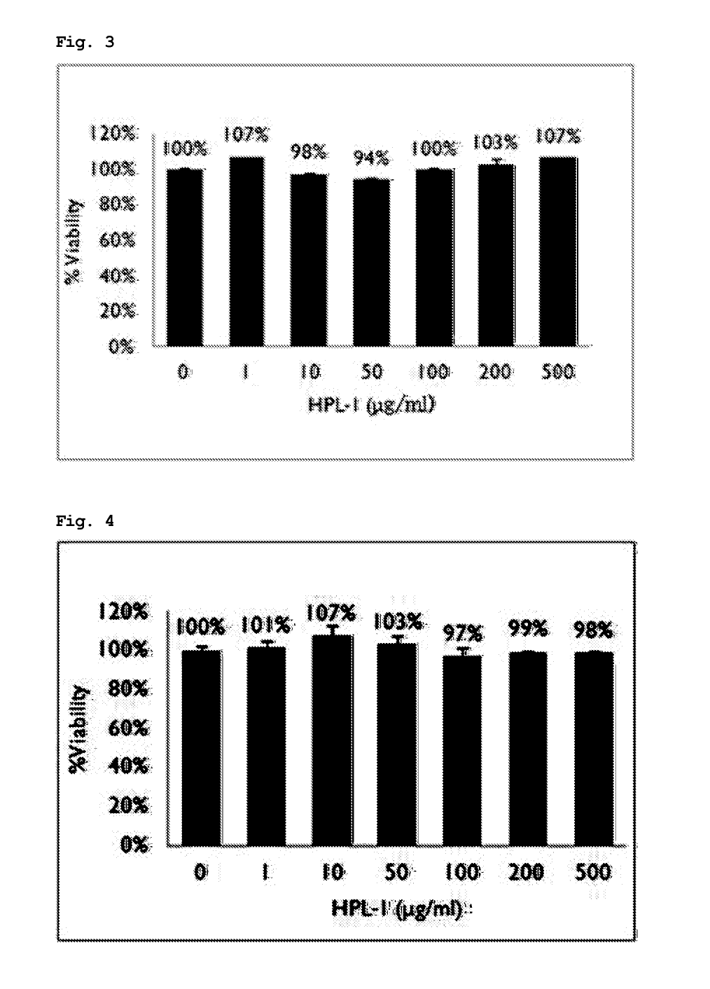

(1) FIG. 3 shows cytotoxicity in synovial sw982 cells incubated with HPL-1 for 24 hours. FIG. 4 shows cytotoxicity in synovial sw982 cells incubated with HPL-1 for 48 hours.

When the cells were incubated with varying concentrations (1, 10, 50, 100, 200 and 500 μg/ml) of HPL-1 for 24 hours, cytotoxicity did not appear at all the concentrations. In addition, cytotoxicity did not appear in the cells incubated for 48 hours after treatment with HPL-1. Thus, it is believed that HPL-1 can be applied up to a concentration of 500 μg/ml in order to evaluate the effect of HPL-1 on the activity of synovial cells.

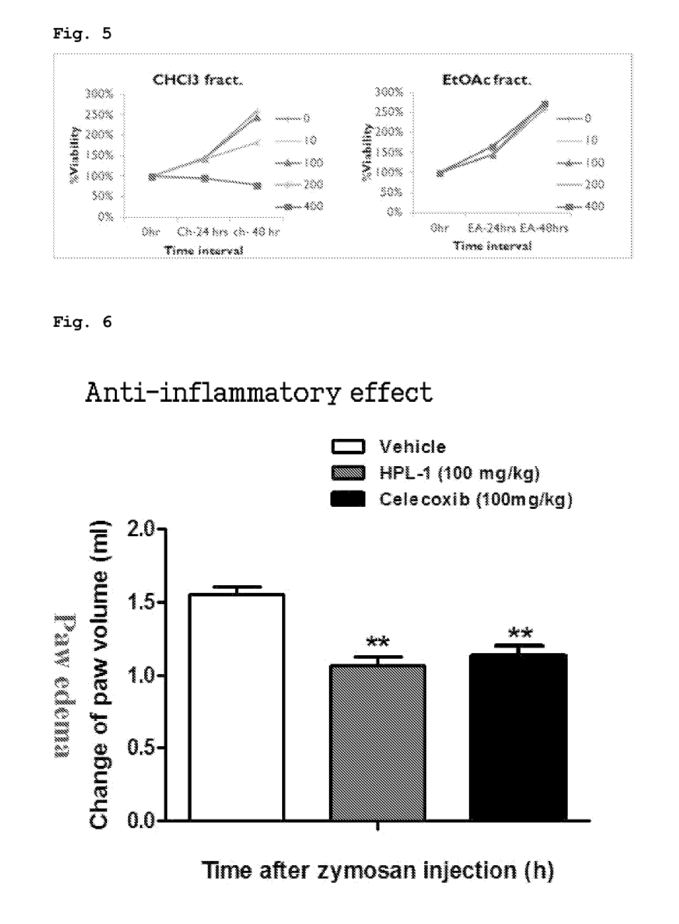

(2) FIG. 5 shows the results of measuring cytotoxicity in a chloroform fraction and ethyl acetate fraction of HPL-1.

As can be seen therein, all the fractions of HPL-1 showed no cytotoxicity at concentrations of 10, 100 and 200 μg/ml, unlike a control, and merely inhibited the proliferation of the cells in a concentration-dependent manner. This suggests that these fractions have the effect of inhibiting the proliferation of inflammatory synovial tissues, which is the symptom of arthritis.

However, when the cells treated with 400 μg/ml of the fractions, the chloroform fraction showed some cytotoxicity with the passage of time, but showed a viability of about 75% even when the cells were incubated for 48 hours.

### TEST EXAMPLE 3

### Anti-Inflammatory Effect Upon Induction of Acute Arthritis in Animal Model

3-1: Preparation for Test

200 μl (20 mg/ml) of zymosan was administered intracutaneously to the right paw of 8-week-old male SD rats to make a zymosan-induced acute arthritis model. This animal model is an animal model similar to a human acute (rheumatoid) arthritis, and entails inflammation and pain.

In this case, zymosan is grey white powder that is a cell wall crude fraction of yeast, and is a mixture including polysaccharides, including glucan (58%) and mannan (18%), proteins, chitins, glycolipids and ash. It is insoluble in water, but is uniformly dispersed in water. It was originally named for the third component of complement. It has received attention as a material that activates the second pathway of complement (properdin-based) and revives the reticuloendothelial system.

3-2: Test Process

The test rats were divided into the following three groups: a group administered with zymosan and vehicle (n=9); a group administered with zymosan and 100 mg/kg of HPL-1 (n=6); and a group administered with zymosan and 100 mg/kg of celexocib (n=6). The group administered with zymosan and celexocib was used as a positive control group.

In each of the test groups, the drugs excluding zymosan were administered orally at 1 hour before administration of zymosan. At 3 hours after the administration of zymosan to the test groups, the effect of inhibiting paw edema was evaluated by observing the volume of rat paw edema.

3-3: Test Results

FIG. 6 is a graph showing the anti-inflammatory effect of HPL-1 upon induction of acute arthritis.

As can be seen in FIG. 6, the volume of edema in the vehicle-administered group was measured to be about 1.5 ml. However, the volume of edema in the group administered with 100 mg/kg of HPL-1 was about 1.0 ml, which was significantly smaller than that in the vehicle-administered group.

In addition, the volume of edema in the group administered with 100 mg/kg of celecoxib was smaller than that in the vehicle-administered group, but greater than that in the group administered with 100 mg/kg of HPL-1.

Thus, it can be seen that HPL-1 had a better anti-inflammatory effect compared to the positive control (celecoxib) that is a drug having the effect of inhibiting edema.

### TEST EXAMPLE 4

### Pain-Relieving Effect Upon Induction of Acute Arthritis in Animal Model

4-1: Preparation for Test

This test was conducted in the same manner Test Example 3-1.

4-2: Test Process

The test rats were divided into the following three groups: a group administered with zymosan and vehicle (n=9); a group administered with zymosan and 100 mg/kg of HPL-1 (n=6); and a group administered with zymosan and 100 mg/kg of celexocib (n=6). The group administered with a combination of zymosan and celexocib was used as a positive control group.

In each of the test groups, the drugs excluding zymosan were administered orally at 1 hour administration of zymosan. During 5 hours after the administration of zymosan to the test groups, the pain-relieving effect was tested by evaluating the allodynia and hyperalgesia resulting from arthritis induced by administration of zymosan.

A mechanical stimulus was applied to the test groups, and the threshold value of the stimulus upon withdrawal was measured. Also, a thermal stimulus was applied to the test groups, and the withdrawal time was measured. Specifically, the test was based on the principle according to which, as the pain of arthritis in the test group becomes more severe, the threshold value of the stimulus, by which the animals of the test groups withdraw their paw, becomes lower, and the time resisting the stimulus becomes gradually shorter.

In this case, the paw withdrawal latency test is based on the principle according to which the animals of the test groups withdraw their paw due to the allodynia or hyperalgesia resulting from arthritis induced in the paw of the animals when a mechanical stimulus or a thermal stimulus is applied to the paw.

4-3: Test Results

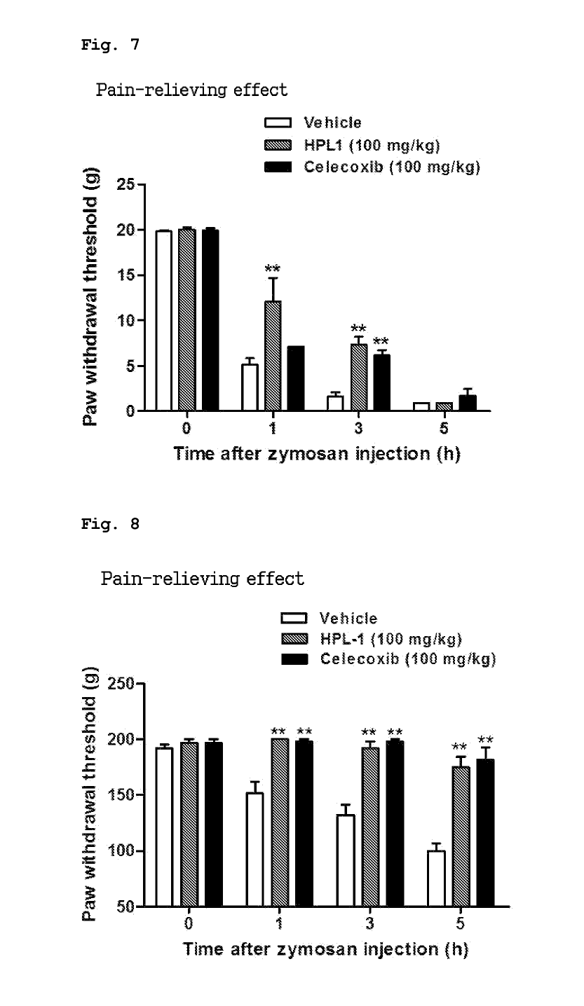

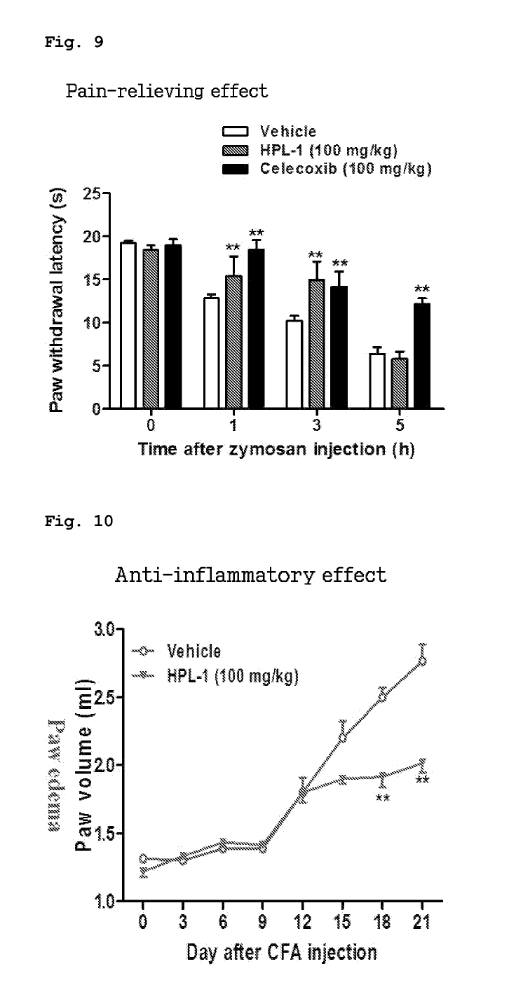

FIG. 7 is a graph showing the pain-relieving effect of HPL-1 in a paw withdrawal latency test for evaluating mechanical allodynia upon induction of acute arthritis; FIG. 8 is a graph showing the pain-relieving effect of HPL-1 in a paw withdrawal latency test for evaluating mechanical hyperalgesia upon induction of acute arthritis; and FIG. 9 is a graph showing the pain-relieving effect of HPL-1 in a paw withdrawal latency test for evaluating thermal hyperalgesia upon induction of acute arthritis.

Referring to FIG. 7, it can be seen that when the mechanical stimulus was applied, the threshold value of the stimulus in all the test groups was reduced over time. This suggests that the pain became more severe with the progression of arthritis.

In addition, the threshold values of the stimulus in the HPL-1-administered group and the celecoxib-administered group were higher than that in the vehicle-administered group, suggesting that HPL-1 and celecoxib all have a pain-relieving effect.

In particular, it can be seen that the pain-relieving effect of HPL-1 after 1 hour and 3 hours was higher than that of the positive control celecoxib.

Referring to FIG. 8, it can be seen that when the mechanical stimulus was applied, the threshold value of the stimulus in the vehicle-administered group was reduced gradually over time.

However, it can be seen that the HPL-1-administered group showed a pain-relieving effect almost similar to that of the celecoxib-administered group.

Referring to FIG. 9, it can be seen that when the thermal stimulus was applied, the paw withdrawal latency in all the test groups was reduced over time.

In addition, it can be seen that the paw withdrawal latency in the HPL-1-administered group and the celecoxib-administered group was shorter than that in the vehicle-administered group, suggesting that HPL-1 and celecoxib all have a pain-relieving effect.

In particular, it can be seen that the pain-relieving effect of HPL-1 after and 3 hours was higher than that of the positive control celecoxib.

### TEST EXAMPLE 5

### Anti-Inflammatory Effect Upon Induction of Chronic Rheumatoid Arthritis in Animal Model

5-1: Preparation for Test

50 μl (20 mg/ml) of CFA (complete Freund's adjuvant [Micobacterium butyricum]) was administered intracutaneously to the right paw of 8-week-old male Lewis rats to prepare a CFA-induced chronic rheumatoid arthritis model, and the anti-inflammatory effect of HPL-1 against edema occurring in the left paw was tested.

5-2: Test Process

The test rats were divided into the following groups: a group administered with CFA and vehicle (n=9); and a group administered with CFA and 100 mg/kg of HPL-1 (n=6). Each of vehicle and HPL-1 was administered orally to the test groups for 3 weeks, and the volume of edema in the left paw was observed.

5-3: Test Results

FIG. 10 is a graph showing the anti-inflammatory effect of HPL-1 upon induction of chronic rheumatoid arthritis.

The group administered with CFA and vehicle was compared with the group administered with CFA and 100 mg/kg of HPL-1, and as a result, the volume of paw edema in the group administered with CFA and vehicle increased greatly over time.

However, it can be seen that the volume of paw edema in the group administered with CFA and 100 mg/kg of HPL-1 increased over time, but the increase in the volume gradually loosened while the rate of the increase gradually decreased.

This suggests that HPL-1 exhibits an anti-inflammatory effect in chronic rheumatoid arthritis and that the anti-inflammatory effect of HPL-1 can further be increased when it is administered at a dose of 100 mg/kg or more.

### TEST EXAMPLE 6

### Pain-Relieving Effect Upon Induction of Chronic Rheumatoid Arthritis in Animal Model

6-1: Preparation for Test

This test was conducted in the same manner as Test Example 5-1.

6-2: Test Process

Test animals were grouped in the same manner as in Test Example 5-2. Vehicle and HPL-1 were orally administered to the test groups while the pain-alleviating effects of HPL-1 against allodynia and hyperalgesia were measured at 3-day intervals.

6-3: Test Results

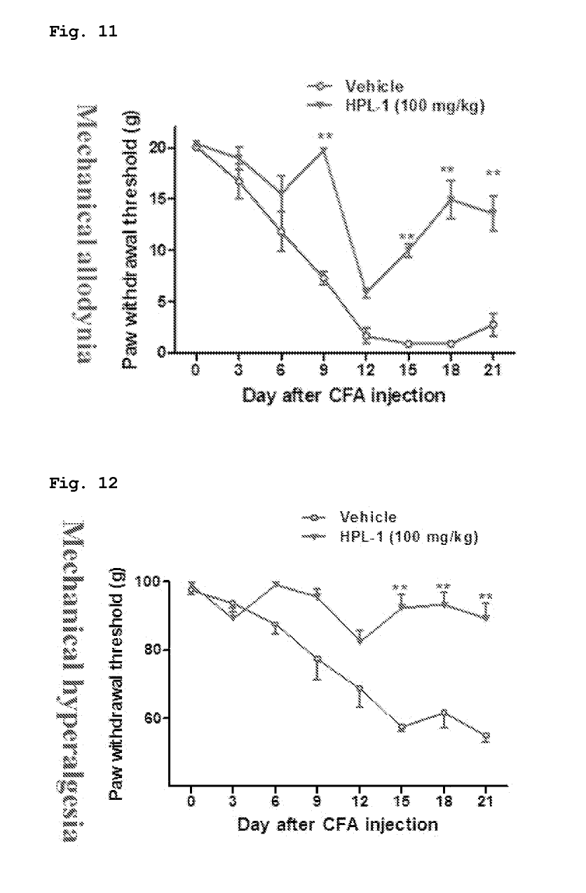

FIG. 11 is a graph showing the pain-relieving effect of HPL-1 in a paw withdrawal latency test for evaluating mechanical allodynia upon induction of chronic rheumatoid arthritis; FIG. 12 is a graph showing the pain-relieving effect of HPL-1 in a paw withdrawal latency test for evaluating mechanical hyperalgesia upon induction of chronic rheumatoid arthritis; and FIG. 13 is a graph showing the pain-relieving effect of HPL-1 in a paw withdrawal latency test for evaluating thermal hyperalgesia upon induction of chronic rheumatoid arthritis.

Referring to FIGS. 11 to 13, it can be seen that the paw withdrawal threshold and the paw withdrawal latency were significantly higher in the HPL-1-administered group than in the vehicle-administered group, suggesting that HPL-1 has a pain-relieving effect against rheumatoid arthritis.

### TEST EXAMPLE 7

### Effect of Alleviating Gait Disturbances Upon Induction of Chronic Rheumatoid Arthritis in Animal Model

7-1: Preparation for Test

The anterior cruciate ligament and medial meniscus of the right knee of 8-week-old SD male rats were removed, and then the rats were forced to move repeatedly to thereby prepare a forced movement-induced chronic (degenerative) osteoarthritis model.

7-2: Test Process

The test rats were divided into the following groups: a Sham group sutured after opening only the capsule of the right knee; a group administered orally with 100 mg/kg of HPL-1 once a day after the removal surgery; and a group administered with vehicle after the removal surgery. The gait patterns of the test rats were continuously observed for 40 days. In this case, the Sham group was used to eliminate the influence of the surgical procedure.

7-3: Test Results

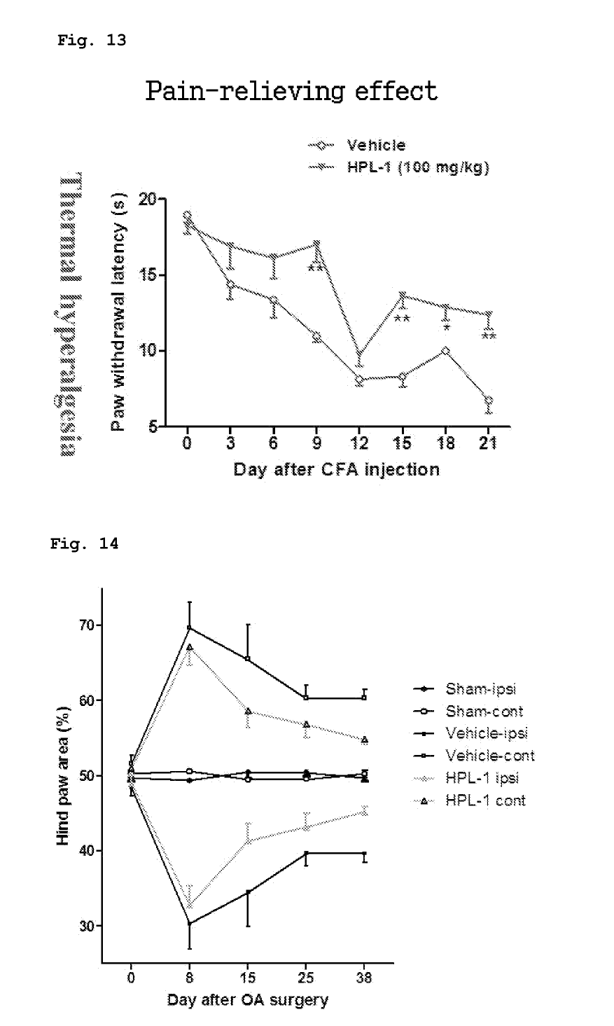

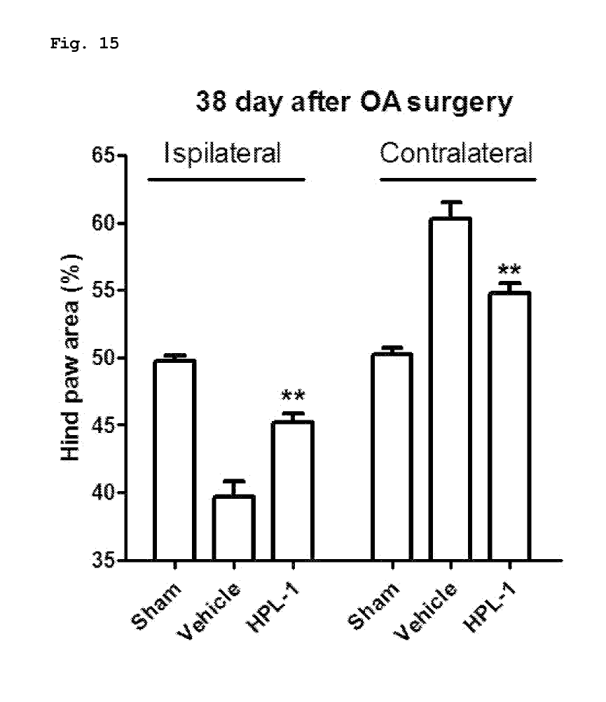

FIGS. 14 and 15 are graphs showing the effect of alleviating gait disturbances upon induction of chronic osteoarthritis. In FIGS. 14 and 15, the term “ipsi” or “ipsilateral” indicates a phenomenon or response that is produced in one side of the body of a bilaterian animal by an action applied to the same side, and the term “cont” or “contralateral” indicates a phenomenon or response that appears in both the side of the body of a bilaterian animal, to which external action was applied, and the opposite side.

Referring to FIGS. 14 and 15, it can be seen that the Sham group showed a normal gait pattern, suggesting that the effect of the surgical procedure on gait disturbances can be eliminated.

Furthermore, it can be seen that the test groups subjected to the removal surgery showed gradual gait disturbance after the surgery, and then showed the most severe gait disturbance.

Moreover, it can be seen that gait disturbances in the vehicle-administered group were partially alleviated between day 8 and day 25 and were not substantially alleviated after day 25, but gait disturbances in the HPL-1-administered group were significantly alleviated continuously between day 8 and day 38.

1. A composition for alleviating or treating rheumatoid arthritis and osteoarthritis, which contains, as an active ingredient, an extract obtained by mixing a first extract, prepared by extracting 350-450 parts by weight of Kalopanacis Cortex, 350-450 parts by weight of Chaenomelis Fructus and 50-150 parts by weight of purified Aconiti Lateralis Preparata Radix, with a second extract prepared by extracting 150-250 parts by weight of Raphani Semen and 50-150 parts by weight of Atractylodis Rhizoma, wherein the extract is not cytotoxic to chondrocytes and synovial cells, and has anti-inflammatory and pain-relieving effects on arthritis patients and an effect of alleviating movement disorders in chronic arthritis patients.

2. The composition of claim 1, wherein an extraction solvent for the first extract is 40-60 wt % alcohol, and an extraction solvent for the second extract is water.

3. The composition of claim 1, wherein a number of times each of the first extract, the second extract and the mixed extract is extracted is two or more.

4-5. (canceled)

6. A dry extract formulation for alleviating or treating rheumatoid arthritis and osteoarthritis, which is prepared by concentrating and drying, contains:
an extract obtained by mixing a first extract, prepared by extracting 350-450 parts by weight of Kalopanacis Cortex, 350-450 parts by weight of Chaenomelis Fructus and 50-150 parts by weight of purified Aconiti Lateralis Preparata Radix, with a second extract prepared by extracting 150-250 parts by weight of Raphani Semen and 50-150 parts by weight of Atractylodis Rhizoma, wherein the extract is not cytotoxic to chondrocytes and synovial cells, and has anti-inflammatory and pain-relieving effects on arthritis patients and an effect of alleviating movement disorders in chronic arthritis patients.

7. The composition of claim 6, wherein an extraction solvent for the first extract is 40-60 wt % alcohol, and an extraction solvent for the second extract is water.

8. The composition of claim 6, wherein a number of times each of the first extract, the second extract and the mixed extract is extracted is two or more.

9. A pharmaceutical formulation for alleviating or treating rheumatoid arthritis and osteoarthritis, which is prepared by adding a pharmaceutically acceptable additive, contains:
an extract obtained by mixing a first extract, prepared by extracting 350-450 parts by weight of Kalopanacis Cortex, 350-450 parts by weight of Chaenomelis Fructus and 50-150 parts by weight of purified Aconiti Lateralis Preparata Radix, with a second extract prepared by extracting 150-250 parts by weight of Raphani Semen and 50-150 parts by weight of Atractylodis Rhizoma, wherein the extract is not cytotoxic to chondrocytes and synovial cells, and has anti-inflammatory and pain-relieving effects on arthritis patients and an effect of alleviating movement disorders in chronic arthritis patients.

10. The composition of claim 9, wherein an extraction solvent for the first extract is 40-60 wt % alcohol, and an extraction solvent for the second extract is water.

11. The composition of claim 9, wherein a number of times each of the first extract, the second extract and the mixed extract is extracted is two or more.
